(19) United States
(12) Patent Application Publication
(10) Pub. No.: US 2016/0331487 A1
(43) Pub. Date: Nov. 17, 2016

(54) PERSONAL ORAL-CARE DEVICE

(71) Applicant: The Procter & Gamble Company, Cincinnati, OH (US)

(72) Inventors: Matthew Lloyd NEWMAN, Cincinnati, OH (US); Kimberly Horn Ellingson, Cincinnati, OH (US); Jayanth RAJAIAH, Loveland, OH (US); Paul Albert SAGEL, MAINEVILLE, OH (US); Franziska SCHMID, FRANKFURT/MAIN (DE)

(21) Appl. No.: 14/713,794

(22) Filed: May 15, 2015

(51) Int. Cl. A61C 1/00

(52) U.S. Cl. CPC A61C 1/0015; A61N 2005/0606

(57) ABSTRACT

A personal oral-care device includes a functional element and a battery supplying energy thereto to cause the functional element to perform at least one oral-care function. The device comprises a body containing the battery; first and second manual switches disposed on the body at a distance from one another, which distance, in combination with a shape of the body, is sufficient to prevent simultaneous activation of the first and second manual switches with a single finger or thumb of a consumer or a single substantially planar surface. The device further includes an electrical circuit structured to activate the functional element only after both the first and second manual switches have been turned on.

### FIELD OF THE INVENTION

The present disclosure is concerned with an oral-care device, and in particular with a personal oral-care electronic device that is structured to prevent its accidental or undesirable activation.

### BACKGROUND OF THE INVENTION

A personal-care electronic device, in particular an electronic device used primarily for oral care, such as, e.g., an electromechanical toothbrush, a light-emitting whitening device, or a heat system for bleaching teeth, must be activated to transfer operation from rest or ‘off’ state to an active or ‘on’ state. Typically, a consumer can activate such devices by a single switch. The switch may comprise a mechanical button providing for completion or closing of an electronic circuit by inducement of physical contact of an otherwise broken conducting path. The switch may also comprise elements structured to detect the occurrence of or absence of inductance, capacitance, external conductance, heat, light, sound (or sub-sonic or ultrasonic vibration), or any external state change. A single switch is usually used because of simplicity, convenience, cost, or industrial-design considerations. One switch may cost less than two or more switches, the likelihood of system failure decreases with complexity and switch count, and the complexity of the user interface may increase with increasing switch count.

Accidental or unintended activation of any mechanical equipment may result in several undesirable events, including, e.g., waste of a battery charge, wear and damage of the device, or even hazard to people. Prevention of such an accidental or unintended activation is therefore important. One method to reduce accidental activation of button-controlled heavy machinery is the use of two buttons linked in the activation circuit by a logic gate, such as, e.g., an AND gate. Such an activation circuit requires two buttons to be pressed simultaneously to activate a primary electrical driving circuit of the machinery. Placement of the two switches at such distance as to be unreachable by two fingers or a finger and a thumb of a single hand ensures that an operator's hands are not in close proximity of the machinery during operation. Alternately, the two switches may be placed in series to complete the conducting path of the activation circuit.

In the context of personal battery-operated oral-care devices, the use of multiple switches to occupy user's hands for safety reasons is not typically necessary. On the contrary, it is highly desirable in most cases to have a system that may be easily and instantaneously activated with a single hand, and most typically with a single finger or thumb, e.g. by pressing a single button. But the ease of the single-button activation of a battery-operated personal oral-care device has its negative sides. First, such a system can be prone to accidental activation, e.g., during packing, transportation, and the like events. That would lead to undesirable draining of the battery power. That may also lead to creating a mechanical problem—if the environment in which the accidental activation has occurred prevents the device from operating as designed. In addition, an accidental activation of a device may be hazardous to people, as it would be, e.g., in the hands of a child who is not physically or mentally prepared to properly and safely handle the device.

Hence, the present disclosure is directed to a battery-operated oral-care device that is conveniently activated with a single hand—and that, at the same time, prevents or at least substantially reduces the likelihood of undesired or accidental activation of the device. In particular, the present disclosure is directed to powered oral-care devices in which the electrical power is provided by either a primary, or non-rechargeable, battery cell—or by a rechargeable battery, and in which the prevention of accidental activation during shipping, storage, accident, or other unintended or undesirable use is important.

### SUMMARY OF THE INVENTION

A personal oral-care device comprises a functional element for performing at least one oral-care function. The device has a body housing a battery, which supplies energy to the functional element. The device has a first manual switch and a second manual switch, both disposed on the body at a distance from one another. The distance between the first and second manual switches, in combination with a shape of the body, is sufficient to prevent simultaneous activation of the first and second manual switches with a single finger or thumb of a consumer or a single substantially planar surface. Depending on the embodiment, the distance between the first manual switch and the second manual switch can be, e.g., between about 5 mm and about 150 mm, between about 10 mm and about 100 mm, between abut 20 mm and about 80 mm, between about 30 mm and about 70 mm, and between about 40 mm and about 70 mm.

An electrical circuit, incorporating therein the first and second manual switches, supplies power from the battery to the functional element. The first and second switches may be connected either in parallel or in series, depending on a particular electrical configuration of the circuit. The electrical circuit is configured to cause activation of the functional element only after both the first manual switch and the second manual switch have been activated by a user.

In one embodiment, at least one of the first manual switch and the second manual switch is recessed within the body sufficiently to prevent an accidental activation of said at least one switch by a substantially planar surface pressing against the body in an area adjacent to said at least one switch. A recess's shape, including a depth, can be configured taking into account ergonomic considerations. For example, the recess may have a radius of curvature or an equivalent thereof of from about 5 mm to about 30 mm. In another embodiment, the recess may have a depth of from about 5 mm to about 30 mm.

The body of the device can have a first side and a second side opposite to the first side. In such an embodiment the first manual switch can be disposed on the first side and the second manual switch can be disposed on the second side. In one embodiment, the first manual switch can be disposed in a first recess formed on the first side of the body, and the second manual switch can be disposed in a second recess formed on the second side of the body. The first recesses may advantageously have at least one dimension (such as length, width, or depth) that is greater than a corresponding dimension of the second recess. Such a configuration may be particularly beneficial in the device intended to be activated by a thumb and one other finger of a user's hand, wherein the first recess can conveniently accommodate the thumb.

In one embodiment of the device, one or both of the first and second manual switches may be structured as a push button configured to be pressed by a user's finger or thumb to activate the device. A finger-contact surface of the first manual switch and/or the second manual switch can have any suitable, preferably ergonomically friendly, shape. As used herein, a finger-contact surface includes a thumb-contact surface. In one embodiment at least one of the first and second manual switches has a concave finger-contact surface. In another embodiment, at least one of the first and second manual switches has a concave finger-contact surface.

In one embodiment, the oral-care device is a tooth-whitening apparatus comprising at least one light source structured and configured to provide illumination of user's front teeth upon activation of the device. Such a tooth-whitening apparatus may include an optical element comprising an interface between the light source and the user's teeth. The optical interface can be advantageously structured and configured to provide a substantially uniform illumination of the user's front teeth upon activation of the device. Examples of the optical interface include transparent or translucent thermoplastics, transparent or translucent thermoplastic elastomers, transparent or translucent silicone rubbers, transparent or translucent glasses, and any combination thereof.

The light source or sources can be any suitable element, e.g., light-emitting diodes, e.g., in the infrared, visible, or ultraviolet range of frequency. Other suitable light sources such as, e.g., lasers in the infrared, visible, or ultraviolet range of frequency can be used as well, in various combinations, depending on the application.

The tooth-whitening apparatus may also include a dental tray structured and configured to receive a tooth-whitening composition therein. The dental tray can have a generally arched shape and configured to fit user's front teeth.

In one embodiment, the tooth-whitening apparatus comprises a timer structured to deactivate the device automatically after a predetermined period of time from activation of the device has expired.

### BRIEF DESCRIPTION OF THE DRAWINGS

While the specification concludes with claims which particularly point out and distinctly claim the subject matter that is regarded as the invention, the various embodiments will be better understood from the following description taken in conjunction with the accompanying drawings, in which:

FIG. 1 is a schematic perspective view of an embodiment of an oral-care device.

FIG. 2 is a schematic plan view of the device shown in FIG. 1.

FIG. 3 is a schematic plan view of another embodiment of the oral-care device.

FIG. 4 is a schematic plan view of yet another embodiment of the oral-care device.

FIG. 5 is a schematic side view of the device shown in FIG. 4.

FIG. 6 is a schematic plan view of another embodiment of the oral-care device.

FIG. 7 is a schematic diagram of an embodiment of an electrical circuit that can be used in the device, wherein switches are in parallel.

FIG. 8 is a schematic diagram of an embodiment of an electrical circuit that can be used in the device, wherein switches are in series.

### DETAILED DESCRIPTION OF THE INVENTION

A personal oral-care device 10 of the disclosure comprises a functional element 20 and a battery 30 supplying energy to the functional element 20 to cause the functional element 20 to perform at least one oral-care function. The personal oral-care device 10 may comprise any electrical or electromechanical device, such as, e.g., a light-emitting tooth-whitening device, a heat source for bleaching teeth, and the like. The at least one oral-care function can be selected from any known operations of these exemplary devices, such as, e.g., tooth brushing, tooth whitening, tooth bleaching, and the like. In one exemplary embodiment, described herein in detail, the device 10 comprises a tooth-whitening apparatus. The functional element 20 of the tooth-whitening apparatus includes at least one light source structured and configured to provide illumination of user's front teeth upon activation of the device 10.

Light-whitening apparatuses are known in the art. Examples include: U.S. Pat. No. 6,416,319 B1, directed to a device for tooth whitening that has a light source; U.S. Pat. No. 7,572,124 B2, directed to an arrangement for use in whitening a patient's teeth; US 2006/0019214 A1, directed to a compact tooth whitening device; US 2010/0086891 A1, directed to a tooth-whitening apparatus; US 2010/0151407 A1, directed to mouthpieces having activated textured surfaces; and US 2012/0009540 A1, directed to a tooth-whitening device including at least one LED and a removable mouthpiece.

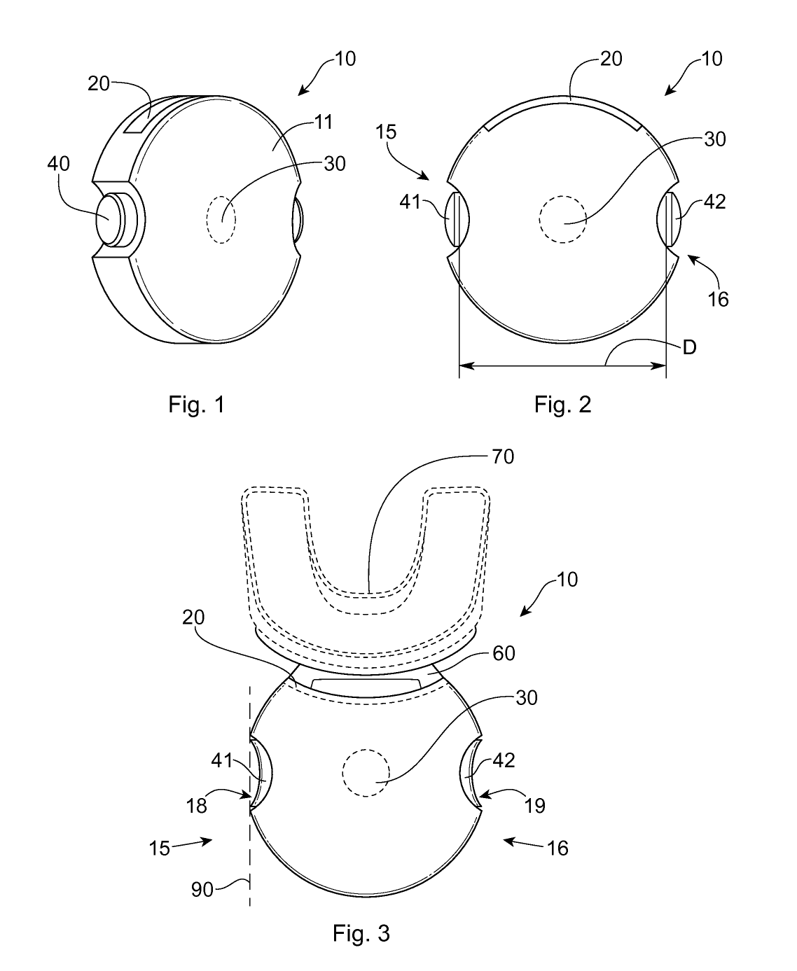

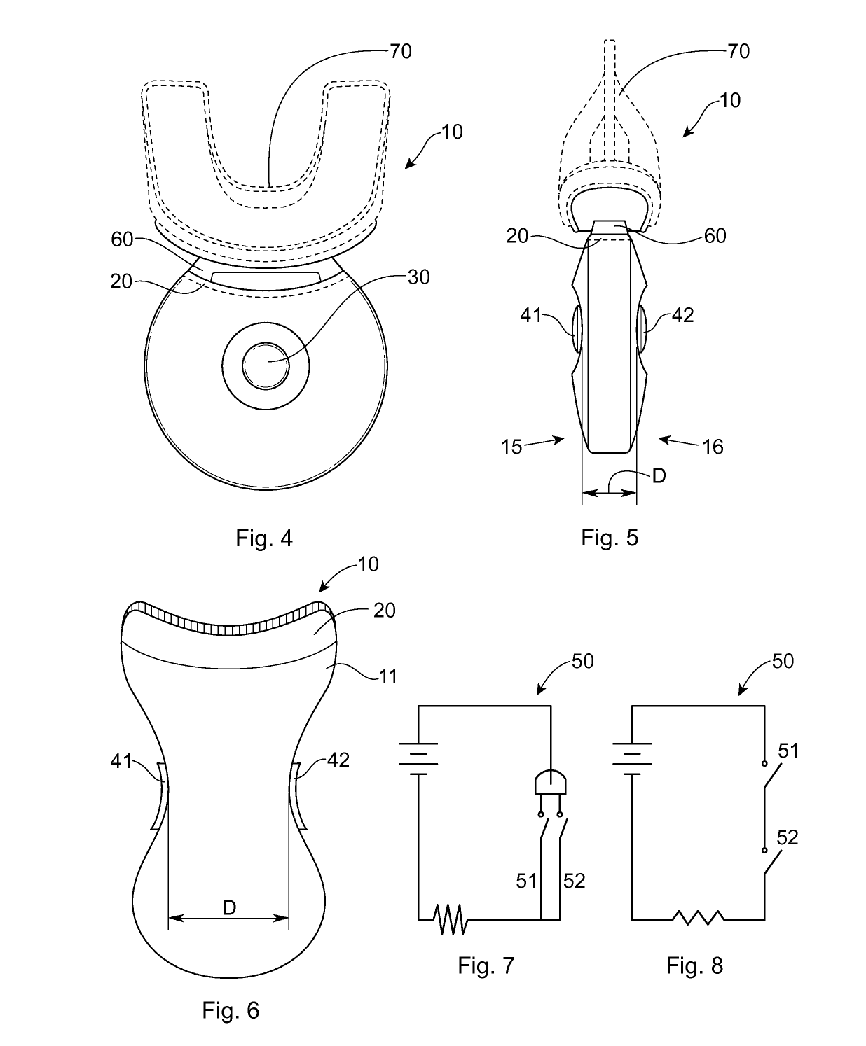

As is shown in FIGS. 1-4, an embodiment of the device 10 comprises a body 11 that houses the battery 30 therein. The device 10 includes at least two manual switches 40 disposed on the body: a first manual switch 41 and a second manual switch 42. The switches 41, 42 are disposed at a distance D from one another. This distance D, in combination with a shape of the body 11, is calculated to prevent simultaneous activation of the first and second manual switches 41, 42 with a single finger or thumb of a user or a single substantially planar surface 90 (FIG. 3). This is designed to avoid accidental or unintended activation of the device 10. In one embodiment of the device, the distance D between the first manual switch 41 and the second manual switch 42 is from 10 mm to 100 mm. In another embodiment, the distance D between the first manual switch 41 and the second manual switch 42 is from 20 mm to 80 mm. In yet another embodiment, the distance D between the first manual switch 41 and the second manual switch 42 is from 30 mm to 70 mm.

The device 10 includes an electrical circuit 50, supplying power from the battery 30 to the functional element 20. The first and second switches 41 and 42 are naturally part of the electrical circuit 50. Any suitable design of the switches 41, 42 may be used in the device 10. Either switch 41, 42 may comprise, e.g., a mechanical button providing for completion or closing of the electronic circuit 50 by inducement of physical contact of an otherwise-broken conducting path. Either switch 41, 42 may also comprise elements structured and configured to detect the occurrence or absence of inductance, capacitance, external conductance, heat, light, sound, sub-sonic or ultrasonic vibration, or any other change of an external state.

Any one of the switches 41, 42 may be structured as a latching switch or a momentary switch. A latching switch is a switch that requires a single, temporary activation by a user to change state permanently, but reversibly, from the ON (or closed) state to the OFF (or open) state. One example of a latching switch is the common household light switch used to open and close main power (110 VAC in the US and 220 VAC in Europe) to a circuit comprising a conducing path and one or more light bulbs. A latching switch is typically a simple mechanical system that usually requires no additional electronic elements for control or timing.

A momentary switch is a switch that closes a conducting path only temporarily during some user-activated state. It is often used for convenience, cost, or industrial-design considerations where a latching switch may not be desired. A momentary switch may, e.g., comprise a button that leaves a conducting path in an electronic circuit open during the “up” position and closes said conducting path when depressed, or during the “down” position. One example of a momentary switch is an elevator button, which, when depressed, completes the conducting path comprising a small ring of light around the button of the desired floor. The lighted ring remains lit even when the button is released by the user. Such a switch may be combined with transistors, microcontrollers, and other electronic elements to provide for continued operation of an electronic device even when said switch is released to the “up” position. In this case, the momentary switch controls an activating circuit which serves only to initiate—but not continue driving—the primary electrical driving circuit. Advantages of a momentary switch are cost and simplicity of the individual switch component design; however, in a system whose primary electrical driving circuit must be active for more than a moment, or must be active while the switch is open, the electronics necessary to connect the activating circuit to the primary electrical driving circuit typically outweigh the cost or simplicity advantages of a momentary switch.

A primary disadvantage of both momentary and latching switches that are designed to be activated by a user is that they can be turned on accidentally—which often leads to an unintended activation of the driving electrical circuit and consequently of the functional element controlled by the driving electrical circuit. For example, the button on an electromechanical toothbrush may be accidentally depressed during placement of the toothbrush into a loose storage in a bathroom drawer. As further examples, the activation button on a light-emitting device may be depressed during rough handling during shipment, or as a result of dropping or placing of the device onto, e.g., a bathroom-counter surface, such that an impact resulting from a contact between the activation button and the surface causes activation of the device. Occurrences like these are highly undesirable, for they may lead to unintended and wasteful battery consumption.

Furthermore, accidental or unintended activation of some electromechanical devices may cause discomfort, particularly for children. For example, the intense light emitted by the tooth-whitening device may, in some instances, be uncomfortable to the human eyes, and particularly the eyes of a child—who may not instantaneously appreciate the discomfort of a prolonged exposure to such a light. In cases where ultraviolet light, in particular, is emitted from a device, safety is of a paramount concern, as the human eye cannot detect the presence of UV light, yet the eye can be damaged by such light. Then, features to mitigate accidental discharge of UV light must be required to ensure safe use. In such instances, a separate system including light in the visible range should be included in parallel with the UV-emitting system, to indicate UV discharge when the device is in the “ON” state.

Accidental activation of switches is not confined to those switches activated by mechanical buttons. Switches may be activated by the occurrence of or absence of inductance, capacitance, external conductance, heat, light, sound (or sub-sonic or ultrasonic vibration), or any external-state change. The touch screen on a modern cellphone is typically an array of capacitive elements activating switches, said capacitive elements sensitive to both intended activation by touch of a finger or thumb, or accidental activation by touch of an earlobe during hand-held conversational use.

The electrical circuit 50 can be structured and configured to conduct the current path that drives the functional element 20 of the device 10. The electrical circuit 50 can comprise, e.g., an electromechanical drive system of a toothbrush (not shown), or to cause a light emission from light-emitting diodes (LEDs) of a tooth-whitening device (FIGS. 1-4), or to cause infrared heat generation in a heat system of a tooth-bleaching device. Any suitable configuration of the electrical circuit 50 can be used in the device 10. For example, FIG. 7 shows an embodiment wherein the first and second switches 41, 42 are included in parallel, while FIG. 8 shows an embodiment wherein the switches 41, 42 are included in series. Regardless of the particular electrical configuration, the device 10 can be activated only when both the first switch 41 and the second switch 42 are in the state of simultaneous activation prompted by a user of the device 10. By “the state simultaneous activation” it is meant that both the first and second switches 41, 42 are caused to be in an ON state at any fraction of time, although the first switch 41 can be initiated or activated (i.e., touched or pressed) in a first instant and the second switch 42 can be initiated or activated (i.e., touched or pressed) in a second instant different in time from the first instant.

At least one of the two manual switches 41, 42 can be advantageously recessed within the body 11 sufficiently to prevent an accidental activation of said at least one switch by a substantially planar surface 90 pressing against the body in an area adjacent to said at least one switch. Such a configuration may be particularly beneficial in situations when the device is expected to be placed on a surface, such as, e.g., a surface of a bathroom counter, and the like, or accidentally dropped on the floor. If the switch 40 is recessed within the body 11 sufficiently to avoid contact with the flat surface when the flat surface contacts the body surrounding the switch 40, accidental activation of the button is virtually excluded.

In the embodiment of FIG. 3, the first manual switch 41 is disposed in a first recess 18 formed on the first side 15 of the body 11, and the second manual switch 42 is disposed in a second recess 19 formed on the second side 16 of the body 11. The first recesses 18 may advantageously have a suitable shape and at least one dimension (such as length, width, or depth) that is greater than a corresponding dimension of the second recess 19. Such a configuration may be particularly beneficial in the device 10 intended to be activated by a thumb and one other finger of a user's hand, wherein the first recess can conveniently accommodate the thumb.

One skilled in the art would be able to envision any number of specific embodiments of the device 10 having two manual switches 40 as is disclosed herein. The device 10 may have, e.g., a first side 15 and a second side 16 opposite the first side 15, so that the first manual switch 41 is disposed on the first side 15 and the second manual switch 42 is disposed on the second side 16, FIGS. 2, 5. In both exemplary embodiments of FIGS. 2, 5, each of the first manual switch 41 and the second manual switch 42 is shown as a push button structured and configured to be pressed or otherwise moved by a user's finger or thumb to activate the device 10. But one skilled in the art will readily recognize that any suitable design of the manual switch can be used in the device 10, e.g., touch-screen buttons and switches comprising capacitive elements.

In further embodiments of the device 10, the manual switches 41, 42 may have any finger-contact surface, preferably ergonomically friendly and/or aesthetically pleasing to a user of the device. For example, at least one of the manual switches 41, 42 may have a finger-contact surface that is concave (FIGS. 3, 6) or convex (FIGS. 2, 5).

The tooth-whitening apparatus may further have an optical interface 60 located between the light source and the user's teeth and structured to provide a substantially uniform illumination of the user's front teeth upon activation of the device. The optical interface can comprise, e.g., transparent or translucent thermoplastics, transparent or translucent thermoplastic elastomers, transparent or translucent silicone rubbers, transparent or translucent glasses, and any combination thereof, as is known in the art. The optical interface may be an integral part of the device 10 or may be incorporated in a replaceable mouthpiece 70, FIG. 3, 4. The mouthpiece 70 can be structured and configured to assist a user in proper positioning of the device relative to the user's teeth and in giving the optical interface a correct orientation relative the user's teeth for a maximal whitening effect. The mouthpiece 70 may also comprise a dental tray 70 and may conveniently have a working surface having a generally arched shape to approximately match the overall shape of the front teeth. The dental tray 70 may also be structured to contain a tooth-whitening composition, as is known in the art. The tooth-whitening composition may be a strip, a gel, or paste.

In a further embodiment, the device 10 can be equipped with a timer structured to deactivate the device 10 automatically after a predetermined period of time from the activation of the device.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal oral-care device comprising a functional element and a battery supplying energy thereto to cause the functional element to perform at least one oral-care function, the device comprising:
a body containing the battery;
a first manual switch disposed on the body;
a second manual switch disposed on the body and at a distance from the first manual switch, wherein the distance between the first and second manual switches, in combination with a shape of the body, is sufficient to prevent simultaneous activation of the first and second manual switches with a single finger or thumb of a consumer or a single substantially planar surface;
and an electrical circuit supplying power from the battery to the functional element and including the first and second manual switches,
wherein the electrical circuit is configured to cause activation of the functional element only after both the first manual switch and the second manual switch have been caused to be in a state of simultaneous activation.

2. The device of claim 1, wherein at least one of the first manual switch and the second manual switch is recessed within the body sufficiently to prevent an accidental activation of said at least one switch by a substantially planar surface pressing against the body in an area adjacent to said at least one switch.

3. The device of claim 1, wherein the body has a first side and a second side opposite the first side, and wherein the first manual switch is disposed on the first side and the second manual switch is disposed on the second side.

4. The device of claim 1, wherein at least one of the first manual switch and the second manual switch comprises a push button structured and configured to be pressed by a user's finger or thumb to activate the device.

5. The device of claim 1, wherein at least one of the first manual switch and the second manual switch comprises a finger-contact surface that is concave.

6. The device of claim 1, wherein at least one of the first manual switch and the second manual switch comprises a finger-contact surface that is convex.

7. The device of claim 1, wherein the first manual switch and the second manual switch are connected in series.

8. The device of claim 1, wherein the first manual switch and the second manual switch are connected in parallel.

9. The device of claim 1, wherein at least one of the first and second manual switches is disposed in a recess formed in the body, the recess having a radius of curvature or an equivalent thereof of from about 5 mm to about 30 mm.

10. The device of claim 1, wherein the first manual switch is disposed in a first recess formed on a first side of the body, and the second manual switch is disposed in a second recess formed on a second side of the body, the first side substantially opposite to the second side, and wherein the first recesses has at least one dimension that is greater than a corresponding dimension of the second recess.

11. The device of claim 10, wherein the at least one dimension is selected from the group consisting of depth, length, and width of the recess.

12. The device of claim 1, wherein the device comprises a tooth-whitening apparatus comprising at least one light source structured and configured to provide illumination of user's front teeth upon activation of the device.

13. The device of claim 12, wherein the tooth-whitening apparatus further comprises an optical interface between the light source and the user's teeth, the optical interface being structured and configured to provide a substantially uniform illumination of the user's front teeth upon activation of the device.

14. The device of claim 12, wherein the light source is selected from the group consisting of light-emitting diodes, in the infrared, visible, or ultraviolet range of frequency; lasers in the infrared, visible, or ultraviolet range of frequency, and any combination thereof.

15. The device of claim 13, wherein the optical interface is selected from the group consisting of transparent or translucent thermoplastics, transparent or translucent thermoplastic elastomers, transparent or translucent silicone rubbers, transparent or translucent glasses, and any combination thereof.

16. The device of claim 12, wherein the tooth-whitening apparatus further comprises at least one dental tray having a generally arched shape, the dental tray being structured to contain a tooth-whitening composition and configured to fit user's front teeth.

17. The device of claim 12, wherein the tooth-whitening apparatus comprises a timer structured to deactivate the device automatically after a predetermined period of time from activation of the device.

18. The device of claim 1, wherein the distance between the first manual switch and the second manual switch is from 10 mm to 100 mm.

19. The device of claim 1, wherein the distance between the first manual switch and the second manual switch is from 20 mm to 80 mm.

20. The device of claim 1, wherein the distance between the first manual switch and the second manual switch is from 30 mm to 70 mm.
